(19) United States
(12) Patent Application Publication
(10) Pub. No.: US 2006/0210521 A1
(43) Pub. Date: Sep. 21, 2006

(54) Composition containing a urea compound and an alkyl glycol carboxylate compound

(75) Inventor: Odile Aubrun-Sonneville, Antony (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/365,527

(22) Filed: Mar. 2, 2006

Related U.S. Application Data
(60) Provisional application No. 60/665,391, filed on Mar. 28, 2005.

(30) Foreign Application Priority Data
Mar. 17, 2005 (FR) 05 50684

(51) Int. Cl. A61K 8/40; A61K 8/365

(52) U.S. Cl. 424/70.22

(57) ABSTRACT

The invention relates to a cleansing composition containing, in an aqueous medium, (1) at least one anionic surfactant chosen from alkyl glycol carboxylic acids and salts thereof, and (2) at least one compound of formula (I) below: 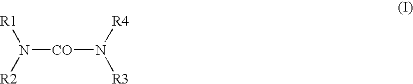 The compound of formula (I) is preferably hydroxyethylurea. The invention also relates to the use of the composition as a cleansing and/or makeup-removing product for the skin, as a shower product, as a shampoo or hair conditioner, as a shaving product or as an exfoliant product.

### REFERENCE TO PRIOR APPLICATIONS

This application claims priority to U.S. provisional application 60/665,391 filed Mar. 28, 2005, and to French patent application 0550684 filed Mar. 17, 2005, both incorporated herein by reference.

### FIELD OF THE INVENTION

The invention relates to a composition containing, in an aqueous medium, a hydroxy urea compound and a particular anionic surfactant, and to its uses for cleansing and/or removing makeup from human skin, the lips and/or the hair.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Cleansing of the skin is very important for facial care, and it should be as efficient as possible since greasy residues such as excess sebum, residues of cosmetic products used daily and makeup products accumulate in the folds of the skin and can block the skin pores and lead to the formation of spots. One good means of cleansing the skin is to use foaming cleansing products. It is generally sought to have products that give a large volume of foam, which is a sign of good cleansing efficacy, and certain surfactants, for instance alkyl glycol carboxylates, are known to give good-quality foams.

It is also sought to have comfortable products, which do not leave a sensation of tautness or dryness.

In point of fact, one of the major difficulties in the formulation of foaming products consists in developing products that have very good foaming power to ensure good cleansing, and which can be rinsed off easily, while at the same time respecting the skin barrier, i.e. without drying but the skin.

One means for improving the sensation of comfort and moisturization is to add moisturizers such as glycerol. However, this type of derivative has a tendency to impair the foaming performance of the products and in particular to reduce the volume of foam obtained.

There is thus still a need for cosmetic cleansing compositions that do not dry out the skin but nevertheless have good foaming power and good rinseability.

### SUMMARY OF THE INVENTION

The inventor has discovered, surprisingly, that it is possible to obtain cleansing products, which provide a moisturizing effect, without compromising foam quality, in particular without impairing the volume of foam, by combining at least one alkyl glycol carboxylate derivative with a urea derivative.

### DETAILED DESCRIPTION OF THE PREFERRED EMBODIMENTS

Thus, one subject of the invention is a composition, preferably suited for and used for cosmetic cleansing, comprising, in an aqueous medium:

(1) at least one anionic surfactant chosen from alkyl glycol carboxylic acids and salts thereof, and

(2) at least one compound of formula (I) below: 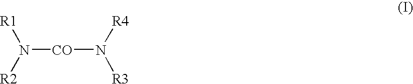
in which:
R1, R2, R3 and R4 represent, independently of each other, a hydrogen atom, a C1-C4 alkyl group or a C2-C6 hydroxyalkyl group containing from 1 to 5 hydroxyl groups, in which at least one of the radicals R1 to R4 represents a hydroxyalkyl group, and also the salts, solvates and isomers thereof.

The composition of the invention has the advantage of not drying out the skin while at the same time having good foaming power and being rinseable. Without being bound by a particular theory, it is believed that the fact that an alkyl glycol carboxylic acid and/or a salt thereof is used as surfactant, in combination with a compound of formula (I) as moisturizer, makes it possible to have a product that gives a foam of very good quality while at the same time having good moisturizing power. Such a result is not obtained in the absence of surfactant of the alkyl glycol carboxylic acid type.

The term “aqueous medium” as used herein means a medium containing water and optionally one or more water-soluble organic solvents. In this aqueous medium, the amount of water is preferably at least 20% by weight and it preferably ranges from 20% to 95% by weight, and better still from 30% to 90% by weight and even better still from 40% to 85% by weight relative to the total weight of the composition.

In addition, since the composition of the invention is preferably a cosmetic composition, it preferably contains a physiologically acceptable medium. The term “physiologically acceptable medium” means herein a medium that is compatible with the skin, mucous membranes and the eyes. It is preferably a cosmetically acceptable medium, i.e. a medium that also has a pleasant colour, odour and feel and that does not cause any unacceptable discomfort (stinging, tautness or redness) liable to put the consumer off using this composition.

Advantageously, the pH of the aqueous medium is compatible with keratin materials and especially with the skin. This pH preferably ranges from 3 to 8.5, better still from 3.5 to 8 and preferentially from 5 to 7.5.

The cleansing composition of the invention is a foaming composition that is preferably rinsed off after application to the skin.

Hydroxy Urea Compound

The at least one urea compound present in the composition according to the invention is a compound of formula (I) below: 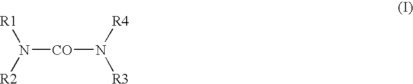
in which:
R1, R2, R3 and R4 represent, independently of each other, a hydrogen atom, a C1-C4 alkyl group or a C2-C6 hydroxyalkyl group containing from 1 to 5 hydroxyl groups, in which at least one of the radicals R1 to R4 represents a hydroxyalkyl group, and also the salts, solvates and isomers thereof.

For the compounds of formula (I):
preferably, R1 denotes a C2-C6 hydroxyalkyl group and R2, R3 and R4 denote, independently of each other, a hydrogen atom or a C1-C4 alkyl group;
preferentially, R1 denotes a C2-C6 hydroxyalkyl group comprising from 1 to 5 hydroxyl groups and especially 1 hydroxyl group, and R2, R3 and R4 denote a hydrogen atom;
more preferentially, R1 denotes a C2-C4 hydroxyalkyl group comprising one hydroxyl group and R2, R3 and R4 denote a hydrogen atom.

C1-C4 alkyl groups that may especially be mentioned include methyl, ethyl, n-propyl, isopropyl, n-butyl, isobutyl and tert-butyl groups.

Among the C1-C6 hydroxyalkyl groups that are preferred are those containing only one hydroxyl group and in particular hydroxyethyl, hydroxypropyl, hydroxybutyl, hydroxypentyl and hydroxyhexyl groups.

Salts of such compounds that may be mentioned include the salts of mineral acids, such as sulfuric acid, hydrochloric acid, hydrobromic acid, hydriodic acid, phosphoric acid and boric acid. Mention may also be made of the salts of organic acids, which may comprise one or more carboxylic acid, sulfonic acid or phosphonic acid groups. They may be linear, branched or cyclic aliphatic acids or alternatively aromatic acids. These acids may also comprise one or more hetero atoms chosen from O and N, for example in the form of hydroxyl groups. Mention may especially be made of propionic acid, acetic acid, terephthalic acid, citric acid and tartaric acid.

The term “solvate” means a stoichiometric mixture of the said compound of formula (I) with one or more water molecules or organic solvent molecules, such as a mixture derived from the synthesis of the compound of formula (I).

Preferred compounds of formula (I) that may be mentioned include N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-[tris(hydroxymethyl)methyl]urea; N-ethyl-N′-(2-hydroxyethyl)urea; N,N-bis(2-hydroxyethyl)urea; N,N′-bis(2-hydroxyethyl)urea; N,N-bis(2-hydroxypropyl)urea; N,N′-bis(2-hydroxypropyl)urea; N,N-bis(2-hydroxyethyl)-N′-propylurea; N,N-bis(2-hydroxypropyl)-N′-(2-hydroxyethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′N′-tetrakis(2-hydroxyethyl)urea; N′N′-bis(2-hydroxyethyl)-N′,N′-bis(2-hydroxypropyl)urea; and mixtures thereof.

Preferably, the compound of formula (I) is N-(2-hydroxyethyl)urea.

The compounds of formula (I) are known compounds and are described especially in patent application DE-A-2 703 185. Among these, N-(2-hydroxyethyl)urea is also commercially available, in the form of a mixture at 50% by weight in water, from the company National Starch under the trade name Hydrovance®.

The compound of formula (I) may be present in the composition according to the invention in any amount (of active material) ranging, for example, from 0.1% to 50% by weight, preferably ranging from 0.1% to 20% by weight and preferentially ranging from 0.1% to 10% by weight relative to the total weight of the composition.

Anionic Surfactant

The composition according to the invention comprises at least one anionic surfactant chosen from alkyl glycol carboxylic acids (or 2-(2-hydroxyalkyloxy)acetic acids), and salts thereof. These surfactants are foaming surfactants.

These surfactants may especially be chosen from compounds of formula (II) below:
R₁—CHOH—CH₂—O—COO⁻X⁺ (II)
in which R₁ denotes a saturated or unsaturated, linear or branched alkyl radical containing from 8 to 30 carbon atoms and X denotes hydrogen or a mineral or organic cation, such as those derived from an alkali metal (for example Na⁺, K⁺), NH₄⁺, ammoniums derived from basic amino acids such as lysine, arginine, sarcosine, ornithine or citrulline, or alternatively amino alcohols such as monoethanolamine, diethanolamine, triethanolamine, glucamine, N-methylglucamine and 3-amino-1,2-propanediol.

The 2-hydroxyalkylcarboxylic acids that are preferred according to the present invention are compounds of formula (II) in which R₁ more particularly denotes a saturated or unsaturated, linear or branched alkyl radical containing from 8 to 18 carbon atoms.

Among the surfactants of formula (II) that may especially be mentioned are sodium lauryl glycol carboxylate, sold under the names Beaulight SHAA® or Beaulight LCA-25N® by the company Sanyo, or the corresponding acid form thereof sold under the name Beaulight SHAA (Acid Form)® by the company Sanyo.

The amount (as active material) of anionic surfactants of alkyl glycol carboxylic type is not limited and may range, for example, from 0.1% to 50% by weight and better still from 1% to 15% by weight relative to the total weight of the composition.

Other Foaming Surfactants

The composition may contain one or more other foaming surfactants, which may be chosen from nonionic, amphoteric or zwitterionic., and anionic foaming surfactants. The amount (as active material) of these other foaming surfactants may range, for example, from 0.1% to 20% by weight and better still from 0.5% to 15% by weight relative to the total weight of the composition.

As nonionic foaming surfactants, the composition may contain, for example, one or more nonionic surfactants chosen from alkyl polyglucosides (APG), maltose esters, polyglycerolated fatty alcohols, and glucamine derivatives, for instance 2-ethylhexyloxycarbonyl-N-methylglucamine, and mixtures thereof.

Alkylpolyglucosides that are preferably used include those containing an alkyl group containing from 6 to 30 carbon atoms and preferably from 8 to 16 carbon atoms, and containing a hydrophilic group (glucoside) preferably comprising 1.2 to 3 glucoside units. Examples of alkylpolyglucosides that may be mentioned include decylglucoside (Alkyl-C9/C11-polyglucoside (1.4)) for instance the product sold under the name Mydol 10® by the company Kao Chemicals, the product sold under the name Plantaren 2000 UP® by the company Cognis, and the product sold under the name Oramix NS 10® by the company SEPPIC; caprylyl/capryl glucoside, for instance the product sold under the name Oramix CG 110® by the company SEPPIC; lauryl glucoside, for instance the products sold under the names Plantaren 1200 N® and Plantacare 1200® by the company Cognis; cocoglucoside, for instance the product sold under the name Plantacare 818/UP® by the company Cognis; and mixtures thereof.

The maltose derivatives are, for example, those described in document EP-A-566 438, such as O-octanoyl-6′-D-maltose or O-dodecanoyl-6′-D-maltose described in document FR-A-2 739 556.

Examples of polyglycerolated fatty alcohols that may be mentioned include polyglycerolated dodecanediol (3.5 mol of glycerol), this product being sold under the name Chimexane NF® by the company Chimex.

The amphoteric and zwitterionic foaming surfactants may be chosen, for example, from betaine derivatives including amidopropylbetaines, amphoacetates and amphodiacetates, and hydroxylsultaines, and mixtures thereof.

Examples of betaine derivatives that may be mentioned include cocobetaine, for instance the product sold under the name Dehyton AB-30® by the company Cognis; laurylbetaine, for instance the product sold under the name Genagen KB® by the company Clariant; oxyethylenated (10 EO) laurylbetaine, for instance the product sold under the name Lauryl ether (10 OE) betaine® by the company Shin Nihon Rica; oxyethylenated (10 EO) stearylbetaine, for instance the product sold under the name Stearyl ether (10 OE) betaine® by the company Shin Nihon Rica; the cocamidopropyl betaine sold, for example, under the name Velvetex BK 35® by the company Cognis; the undecylenamidopropyl betaine sold, for example, under the name Amphoram U® by the company Ceca; and mixtures thereof.

Examples of amphoacetates and amphodiacetates that may be mentioned include N-disodium N-cocoyl-N-carboxymethoxyethyl-N-carboxymethylethylenediamine (CTFA name: disodium cocamphodiacetate), for instance the product sold under the name Miranol C2M Concentrate NP® by the company Rhodia Chimie; N-sodium N-cocoyl-N-hydroxyethyl-N-carboxymethylethylenediamine (CTFA name: sodium cocamphoacetate), and mixtures thereof.

The anionic surfactants may be chosen, for example, from soaps (alkaline salt of fatty acids), acylamino acids such as acylglycinates, amidoether carboxylates, alkyl polyaminocarboxylates, alkyl ether sulfates such as sodium laureth sulfates, alkyl sulfonates, isethionates, alkyl methyl taurates, alkyl sulfosuccinates, alkyl sulfoacetates, monoalkyl phosphates and dialkyl phosphates, salts thereof, and mixtures thereof.

Adjuvants

As indicated above, the aqueous medium of the composition according to the invention may contain, besides water, one or more water-soluble solvents that may be chosen, for example, from lower alcohols (monoalcohols) containing from 1 to 6 carbon atoms, such as ethanol; and polyols such as glycerol, glycols, for instance butylene glycol, isoprene glycol, propylene glycol, polyethylene glycols such as PEG-8; sorbitol; sugars such as glucose, fructose, maltose, lactose and sucrose; and mixtures thereof, provided that these compounds do not impair the desired properties of the composition according to the invention. The amount of solvent(s) in the composition of the invention may range, for example, from 0.1% to 15% by weight, preferably from 0.5% to 10% by weight, better still from 0.5% to 7.5% by weight and even better still from 0.5% to 5% by weight relative to the total weight of the composition.

The composition according to the invention may also contain any adjuvant or additive usually used in cosmetics. Needless to say, a person skilled in the art will take care to select the optional additive(s) for the composition according to the invention such that the advantageous properties intrinsically associated with the composition in accordance with the invention preferably are not, or are not substantially, adversely affected by the envisaged addition.

Among the conventional adjuvants that may be added, mention may be made of preserving agents; sequestrants (EDTA); antioxidants; fragrances; dyestuffs such as soluble dyes, pigments and nacres; fillers with a matting, tensioning, whitening or exfoliant effect; sunscreens; hydrophilic or lipophilic cosmetic or dermatological active agents, such as vitamins, antiseptics, antiseborrhoeic agents, antimicrobial agents, for instance benzoyl peroxide, salicylic acid, triclosan, azeleic acid, niacin (vitamin PP), slimming agents, for instance caffeine, optical brighteners, electrolytic agents, agents for improving the cosmetic properties of the skin, or spherical or non-spherical, porous or non-porous solid particles of any size. The amounts of these various adjuvants include those conventionally used in the field under consideration, for example from 0.01% to 20% of the total weight of the composition. These adjuvants and the concentrations thereof should be such that they do not modify the property desired for the composition of the invention.

Examples of exfoliants that may be mentioned include exfoliant or scrubbing particles of mineral, plant or organic origin. Thus, it is possible to use, for example, polyethylene beads or powder, Nylon powder, polyvinyl chloride powder, pumice, ground apricot kernel or walnut husk, sawdust, glass beads and alumina, and mixtures thereof. These particles may be present in an amount ranging, for example, from 0.5% to 30% by weight, preferably from 1% to 20% by weight and better still from 1% to 10% by weight relative to the total weight of the composition. When the composition contains exfoliant particles, it may especially constitute a composition for scrubbing facial or body skin.

The compositions may also contain anionic, nonionic, cationic or amphoteric, hydrophilic or lipophilic polymers, thickeners or dispersants. Preferably, these materials are added such that they do not affect the qualities of the composition.

Cationic or amphoteric polymers that may especially be mentioned include those of the Polyquaternium type (CTFA name), which give the foaming cream softness and creaminess. These polymers may preferably be chosen from the following polymers:
Polyquaternium 5, such as the product Merquat 5 sold by the company Calgon;
Polyquaternium 6, such as the product Salcare SC 30 sold by the company Ciba, and the product Merquat 100 sold by the company Calgon;
Polyquaternium 7, such as the products Merquat S, Merquat 2200 and Merquat 550 sold by the company Calgon; and the product Salcare SC 10 sold by the company Ciba;
Polyquaternium 10, such as the product Polymer JR400 sold by the company Amerchol;
Polyquaternium 11, such as the products Gafquat 755, Gafquat 755N and Gafquat 734 sold by the company ISP;
Polyquaternium 15, such as the product Rohagit KF 720 F sold by the company Rohm;
Polyquaternium 16, such as the products Luviquat FC905, Luviquat FC370, Luviquat HM552 and Luviquat FC550 sold by the company BASF;
Polyquaternium 22, such as the product Merquat 280 sold by the company Calgon;
Polyquaternium 28, such as the product Styleze CC10 sold by the company ISP;
Polyquaternium 39, such as the product Merquat Plus 3330 sold by the company Calgon;
Polyquaternium 44, such as the product Luviquat Care sold by the company BASF;
Polyquaternium 46, such as the product Luviquat Hold sold by the company BASF;
Polyquaternium 47, such as the product Merquat 2001 sold by the company Calgon.

Cationic polymers that may also be used include cationic guars, such as the product Jaguar sold by the company Rhodia.

Anionic polymers that may especially be mentioned include those comprising at least one hydrophobic chain, and in particular those derived from acrylic or methacrylic acid, for instance the acrylates/steareth-20 methacrylate copolymer sold under the name Aculyn 22 by the company Rohm & Haas (CTFA name: acrylates/steareth-30 methacrylate copolymer); (meth)acrylic acid/ethyl acrylate/oxyethylenated (25 EO) behenyl methacrylate terpolymer, as anaqueous emulsion sold under the name Aculyn 28 by the company Rohm & Haas: acrylic acid/oxyethylenated (20 EO) monocetyl itaconate copolymer, as an aqueous 30% dispersion sold under the name Structure 3001 by the company National Starch; acrylic acid/oxyethylenated (20 EO) monostearyl itaconate copolymer as an aqueous 30% dispersion sold under the name Structure 2001 by the company National Starch; acrylates/acrylate copolymer modified with polyoxyethylenated (25 EO) C12-C24 alcohols, in the form of a latex containing 30-32% copolymer, sold under the name Synthalen W2000 by the company 3V SA, and also the copolymers sold under the names Pemulen and Carbopol by the company Noveon, for instance acrylate/C₁₀-C₃₀-alkylacrylate copolymer such as the products Pemulen TR1, Pemulen TR2 and Carbopol 1382 (CTFA name: acrylates/C10-30 alkyl acrylate crosspolymer).

The composition may also contain polymers comprising at least one monomer containing a sulfonic group, and especially 2-acrylamido-2-methylpropane-sulfonic acid (AMPS) polymers and copolymers such as:
the crosslinked and neutralized homopolymer of 2-acrylamido-2-methylpropanesulfonic acid sold by the company Clariant under the trade name “Hostacerin AMPS” (CTFA name: ammonium polyacryldimethyltauramide);
crosslinked anionic copolymers of acrylamide or methacrylamide and of 2-acrylamido-2-methylpropanesulfonic acid, for instance those in the form of a W/O emulsion, such as those sold under the name Sepigel 305 by the company SEPPIC (CTFA name: Polyacrylamide/C13-14 isoparaffin/Laureth-7), under the name Simulgel 600 by the company SEPPIC (CTFA name: acrylamide/sodium acryloyldimethyltaurate copolymer/isohexadecane/Polysorbate 80),
copolymers of (meth)acrylic acid or of (meth)acrylate and of 2-acrylamido-2-methylpropanesulfonic acid, especially those in the form of a W/O emulsion, such as those sold under the name Simulgel NS by the company SEPPIC (copolymer of acrylamido-2-methylpropanesulfonate/hydroxyethyl acrylate as a 40% inverse emulsion in Polysorbate 60 and squalane) (CTFA name: hydroxyethyl acrylate/sodium acryloyldimethyltaurate copolymer/squalane/Polysorbate 60) or those sold under the name Simulgel EG by the company SEPPIC (copolymer of acrylic acid/acrylamido-2-methylpropanesulfonic acid in the form of the sodium salt as a 45% inverse emulsion in isohexadecane/water) (CTFA name: sodium acrylate/sodium acryloyldimethyltaurate copolymer/isohexadecane/Polysorbate 80),
copolymers of 2-acrylamido-2-methylpropanesulfonic acid and of vinylpyrrolidone or vinylformamide, such as the products sold under the names Aristoflex AVC by the company Clariant,
hydrophobic-modified AMPS polymers, such as, especially, the copolymer of AMPS and of ethoxylated C12-C14 alkyl methacrylate (non-crosslinked copolymer obtained from Genapol LA-070 and AMPS) (CTFA name: ammonium acryloyldimethyltaurate/Laureth-7 methacrylate copolymer) sold under the name Aristoflex LNC by the company Clariant, and the copolymer of AMPS and of ethoxylated (25 EO) stearyl methacrylate) (copolymer crosslinked with trimethylolpropane triacrylate, obtained from Genapol T-250 and AMPS) (CTFA name: ammonium acryloyldimethyltaurate/Steareth-25 methacrylate crosspolymer) sold under the name Aristoflex HMS by the company Clariant.

These polymers may be in any amount (of active material) ranging, for example, from 0.05% to 10% by weight and better still from 0.1% to 5% by weight relative to the total weight of the composition.

The compositions according to the invention are in the form of solutions, gels, milks or more or less soft creams, and they may be used on any keratin material, such as the skin, the scalp, the hair, the eyelashes, the eyebrows, the nails or mucous membranes, especially as a hygiene product, for example as a product for cleansing the skin, mucous membranes and/or the hair, in particular as a cleansing and/or makeup-removing product for the skin (of the face and/or the body), as a shower product (two-in-one product), as a shampoo, as a hair conditioner, as a shaving product, as a rinse-off mask, as an exfoliant product (also known as a desquamating or scrubbing product), for either the face or the body or the hands, after addition of exfoliant particles.

A subject of the invention is also the cosmetic use of the composition as defined above, as a cleansing and/or makeup-removing product for the skin, as a shower product, as a shampoo or hair conditioner, as a shaving product, as a rinse-off mask or as an exfoliant product.

Another subject of the invention is a process for cleansing a keratin material, such as the skin, the scalp, the hair, the eyelashes, the eyebrows, the nails or mucous membranes, wherein a composition as defined above is applied to the keratin material and is rinsed off.

The keratin material is preferably the skin.

The compositions according to the invention may be used, for example, in the following manner:

1) when the compositions according to the invention are mild cleansing products for the face, they may be used in the following manner:
the product is worked into a lather in the hands with water
the lather is applied to the face,
the face is cleansed,
it is rinsed with water.

2) when the compositions according to the invention are makeup-removing products, they may be used as indicated above, but they may also be applied dry to the face, and then massaged until satisfactory makeup removal has been obtained, and rinsed off with water, or alternatively they may be applied using cotton-wool.

3) They may be used as a two-in-one shower product, as a shampoo and/or hair conditioner, as a rinse-off mask or as a shaving product, in the usual manner for using these products.

The examples that follow serve to illustrate the invention without, however, being limiting in nature. The amounts indicated correspond, unless otherwise mentioned, to the amount of active material and not to the amount of starting material. The names of the compounds used are given as the CTFA name or as the chemical name.

### EXAMPLE 1 ACCORDING TO THE INVENTION AND COMPARATIVE EXAMPLES 1 AND 2

| | Example 1 | | |
|---|---|---|---|
| | according | | |
| | to the | Comparative | Comparative |
| Foaming composition | invention | Example 1 | Example 2 |
| Water | qs 100% | qs 100% | qs 100% |
| N-(2- | 10% | — | 10% |
| Hydroxyethyl)urea | | | |
| (1) | | | |
| Sodium lauryl | 10.4% | 10.4% | — |
| glycol carboxylate | | | |
| (2) | | | |
| Sodium Laureth | | | 10.4% |
| sulfate | | | |
| Glycerol | — | 10% | — |
| Citric acid | 0.1% | 0.1% | 0.1% |
| Preserving agents | 0.4% | 0.4% | 0.4% |
| pH | 7 | 7 | 7.4 |
| Volume of foam 1 | 4.5 | 3.2 | 2.3 |
| Volume of foam 2 | 8.3 | 6.7 | 5 |

(1) i.e. 20% of Hydrovance sold by the company National Starch (hydroxyethyl urea as a 50% by weight aqueous solution)
(2) Beaulight Shaa sold by the company Sanyo
   Procedure: an aqueous solution containing the moisturizer (Hydrovance or glycerol), the surfactant (Beaulight or Laureth sulafte)and the preserving agents was prepared. Then, citric acid was added, and the amount of water was adjusted to 100%.
   Test of Performance of the Compositions:

The foam qualities of the compositions of the examples given above were evaluated according to the following protocol:

Before any use of the products, the hands were washed with household soap and then rinsed and dried thoroughly.
1—wet the hands by passing them under running water, and shake them three times to drain the water off,
2—place 1 g of product in the palm of one of the hands,
3—work the product between the two palms for 10 seconds,
4—evaluate the volume of foam (volume of foam 1),
5—add 2 ml of water and work the product again for 10 seconds,
6—evaluate the volume of foam (volume of foam 2)

The volume was graded on a scale from 0 to 10.

The evaluation panel consisted of three trained experts. The mean of the three grades allowed the products to be compared according to each of the criteria.

The results for the volume of foam 1 and the volume of foam 2 are given in the above table. They show that the example according to the invention provided good moisturization of the skin while at the same time giving a better volume of foam than the comparative example 1 which contained glycerin in the place of Hydrovance, and than the comparative example 2 which contained another anionic surfactant than Beaulight.

### EXAMPLES 2 TO 5 ACCORDING TO THE INVENTION

| Composition | Example 2 | Example 3 | Example 4 | Example 5 |
|---|---|---|---|---|
| Sodium lauryl glycol carboxylate (1) | 7.8 | 3.9 | 7.8 | 3.9 |
| Cocobetaine (2) | 6.5 | 3.25 | — | — |
| Cocoamidopropylbetaine (3) | — | — | 6.5 | — |
| Cocoglucoside (4) | — | — | — | 6.5 |
| Ammonium acryloyldimethyltaurate/ | — | 1 | — | — |
| Stearate-25 methacrylate | | | | |
| crosspolymer (5) | | | | |
| PEG-150 pentaerythrityl tetrastearate (6) | 1 | — | 2 | — |
| Acrylates/C10-30 alkyl acrylate | — | — | — | 1 |
| crosspolymer (7) | | | | |
| Silica (8) | — | — | 5 | — |
| Polyquaternium-7 | 0.3 | — | — | — |
| PEG-14M (9) | — | — | — | 0.5 |
| N-(2-Hydroxyethyl)urea (10) | 10 | 5 | 10 | 10 |
| Citric acid | qs pH 7 | qs pH 7 | qs pH 7 | qs pH 7 |
| DMDM Hydantoin | 0.2 | 0.2 | 0.2 | 0.2 |
| Triethanolamine | — | — | — | 1 |
| Sodium methylparaben | 0.3 | 0.3 | 0.3 | 0.3 |
| Water | qs 100 | qs 100 | qs 100 | qs 100 |

(1) Beaulight Shaa sold by the company Sanyo
(2) Dehyton AB 30 ® sold by the company Cognis (containing 30% by weight of active material)
(3) Tegobetaine F50 ® sold by the company Goldschmidt (containing 38% active material)
(4) Plantacare 818 UP sold by the company Cognis (containing 53% active material)
(5) Aristoflex HMS sold by the company Clariant
(6) Crothix sold by the company Croda
(7) Carbopol 1382 sold by the company Noveon
(8) Aerosil 200 sold by the company Degussa-Huls
(9) Polyox WSR 205 sold by the company Amerchol
(10) Hydrovance sold by the company National Starch (as an aqueous solution containing 50% active material)

### EXAMPLES 6 AND 7 ACCORDING TO THE INVENTION

| Composition | Example 6 | Example 7 |
|---|---|---|
| Sodium lauryl glycol carboxylate (1) | 7.8 | 7.8 |
| Sodium lauryl ether sulfate | 3.25 | — |
| Potassium cocoyl glycinate (2) | — | 3.25 |
| Cocoglucoside (3) | 3.25 | 3.25 |
| PEG-150 pentaerythrityl tetrastearate (4) | 1 | — |
| Acrylates/C10-30 alkyl acrylate crosspolymer (5) | — | 1 |
| PEG-7 glyceryl cocoate | — | 1 |
| Glycol distearate | — | 2 |
| Corn starch | — | 2 |
| N-(2-Hydroxyethyl)urea (6) | 2.5 | 10 |
| Triethanolamine | — | 1 |
| Citric acid | qs pH 7 | qs pH 7 |
| DMDM Hydantoin | 0.2 | 0.2 |
| Sodium methylparaben | 0.3 | 0.3 |
| Water | qs 100 | qs 100 |

(1) Beaulight Shaa sold by the company Sanyo
(2) Amilite GCS12 sold by the company Ajinomoto
(3) Plantacare 818 UP sold by the company Cognis (containing 53% active material)
(4) Crothix sold by the company Croda
(5) Carbopol 1382 sold by the company Noveon
(6) Hydrovance sold by the company National Starch (as an aqueous solution containing 50% active material)

The above written description of the invention provides a manner and process of making and using it such that any person skilled in this art is enabled to make and use the same, this enablement being provided in particular for the subject matter of the appended claims, which make up a part of the original description and including a cleansing composition containing, in an aqueous medium, (1) at least one anionic surfactant chosen from alkyl glycol carboxylic acids and salts thereof, and (2) at least one compound of formula (I) below: 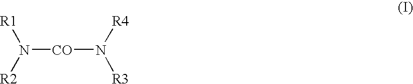
in which:
R1, R2, R3 and R4 represent, independently of each other, a hydrogen atom, a C1-C4 alkyl group or a C2-C6 hydroxyalkyl group containing from 1 to 5 hydroxyl groups, in which at least one of the radicals R1 to R4 represents a hydroxyalkyl group, and also the salts, solvates and isomers thereof.

As used above, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in an aqueous medium:
(1) at least one anionic surfactant chosen from alkyl glycol carboxylic acids and salts thereof, and
(2) at least one compound chosen from formula (I) below: 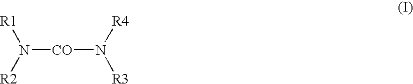
in which:
R1, R2, R3 and R4 represent, independently of each other, a hydrogen atom, a C1-C4 alkyl group or a C2-C6 hydroxyalkyl group containing from 1 to 5 hydroxyl groups, in which at least one of the radicals R1 to R4 represents a C2-C6 hydroxyalkyl group, and the salts and solvates thereof.

2. The composition according to claim 1, wherein it comprises at least 20% by weight of water relative to the total weight of the composition.

3. The composition according to claim 1, comprising at least one compound of formula (I) wherein R1 denotes a C2-C6 hydroxyalkyl group and R2, R3 and R4 denote, independently of each other, a hydrogen atom or a C1-C4 alkyl group.

4. The composition according to claim 1, comprising at least one of formula (I) wherein R1 denotes a C2-C6 hydroxyalkyl group comprising from 1 to 5 hydroxyl groups, and R2, R3 and R4 denote a hydrogen atom.

5. The composition according to claim 4, wherein R1 denotes a C2-C6 hydroxyalkyl group comprising 1 hydroxyl group.

6. The composition according to claim 1, comprising at least one compound of formula (I) wherein R1 denotes a C2-C4 hydroxyalkyl group comprising 1 hydroxyl group and R2, R3 and R4 denote a hydrogen atom.

7. The composition according to claim 1, comprising at least one compound chosen from N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-[tris(hydroxymethyl)methyl]urea; N-ethyl-N′-(2-hydroxyethyl)urea; N,N-bis(2-hydroxyethyl)urea; N,N′-bis(2-hydroxyethyl)urea; N,N-bis(2-hydroxypropyl)urea; N,N′-bis(2-hydroxypropyl)urea; N,N-bis(2-hydroxyethyl)-N′-propylurea; N,N-bis(2-hydroxypropyl)-N′-(2-hydroxyethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′N′-tetrakis(2-hydroxyethyl)urea; N′N′-bis(2-hydroxyethyl)-N′,N′-bis(2-hydroxypropyl)urea; and mixtures thereof.

8. The composition according to claim 1, comprising N-(2-hydroxyethyl)urea.

9. The composition according to claim 1, wherein the salts of the compounds of formula (I) are chosen from the salts of sulfuric acid, hydrochloric acid, hydrobromic acid, hydriodic acid, phosphoric acid, boric acid, propionic acid, acetic acid, terephthalic acid, citric acid and tartaric acid.

10. The composition according to claim 1, wherein the compound(s) of formula (I) is/are present in an amount ranging from 0.1% to 50% by weight relative to the total weight of the composition.

11. The composition according to claim 1, comprising at least one anionic surfactant chosen from compounds of formula (II):
R₁—CHOH—CH₂—O—COO⁻ X⁺ (II)
in which R₁ denotes a saturated or unsaturated, linear or branched alkyl radical containing from 8 to 30 carbon atoms and X denotes hydrogen or a mineral or organic cation.

12. The composition according to claim 11, comprising at least one anionic surfactant that is a compound of formula (II) in which R₁ denotes a saturated or unsaturated, linear or branched alkyl radical containing from 8 to 18 carbon atoms.

13. The composition according to claim 1, comprising sodium lauryl glycol carboxylate and/or the acid form thereof.

14. The composition according to claim 1, wherein the amount of anionic surfactant(s) (1) is 0.1% to 50% by weight relative to the total weight of the composition.

15. The composition according to claim 1, further comprising one or more foaming surfactants chosen from nonionic, amphoteric, and zwitterionic surfactants, and anionic surfactants other than anionic surfactant(s) (1).

16. The composition according to claim 1, wherein it is a cleansing and/or makeup-removing product for the skin, a shower product, a shampoo, a hair conditioner, a shaving product, a rinse-off mask or an exfoliant product.

17. The composition according to claim 1, comprising at least one salt of a compound of formula (I) chosen from the salts of sulfuric acid, hydrochloric acid, hydrobromic acid, hydriodic acid, phosphoric acid, boric acid, propionic acid, acetic acid, terephthalic acid, citric acid and tartaric acid.

18. The composition according to claim 1, comprising at least one solvate of a compound of formula (I).

19. The composition according to claim 1, comprising at least one salt of a compound of formula (I).

20. A process for cleansing a keratin material, wherein-a composition according to claim 1 is applied to the keratin material, and then is rinsed off.
